(19) United States
(12) Patent Application Publication
(10) Pub. No.: US 2026/0041412 A1
(43) Pub. Date: Feb. 12, 2026

(54) MEDICAL DEVICES, SYSTEMS, AND RELATED METHODS FOR COLLECTING TISSUE

(71) Applicant: Boston Scientific Scimed, Inc., Maple Grove, MN (US)

(72) Inventors: Jessica Tova Brand BECHLY, Maple Grove, MN (US); Brenda Joy THEIS, Zimmerman, MN (US); Danika WHALEN, Blaine, MN (US); Kirsten IWANSKI, Maple Grove, MN (US); Margaret ANDERSON, Maple Grove, MN (US); Matthew MORTON, Isanti, MN (US); Riley FRANZO, Canyon Lake, CA (US); Yash SONI, Minneapolis, MN (US); Zachary Oliver Morell NICKLE, Minneapolis, MN (US); Abbylee MAEDER, Hanover, MN (US); Hebert Alejandro QUINTERO RINCON, The Woodlands, TX (US)

(73) Assignee: Boston Scientific Scimed, Inc., Maple Grove, MN (US)

(21) Appl. No.: 19/290,874

(22) Filed: Aug. 5, 2025

Related U.S. Application Data
(60) Provisional application No. 63/681,386, filed on Aug. 9, 2024.

(51) Int. Cl. A61B 10/04; A61B 10/02

(52) U.S. Cl. CPC A61B 10/04; A61B 2010/0208; A61B 2010/0225

(57) ABSTRACT

A medical device includes an outer tube defining a lumen, an inner tube movable within the lumen of the outer tube and an end effector at a distal end of the inner tube. The end effector retracts to capture one or more tissue samples. A capsule is positioned within a distal portion of the inner tube. The capsule includes a proximal opening and a distal opening for receiving the one or more tissue samples.

### CROSS-REFERENCE TO RELATED APPLICATIONS

This application claims the benefit of priority to U.S. Provisional Application No. 63/681,386, filed on Aug. 9, 2024, which is incorporated by reference herein in its entirety.

### TECHNICAL FIELD

This disclosure generally relates to medical devices, systems, and related methods for collecting tissue. More specifically, aspects of this disclosure pertain to internal components of a shaft, such as capsules, useful for collecting tissue samples, such as biopsy samples, within the body of a subject.

### BACKGROUND

Medical devices are often inserted into the body to perform a therapeutic and/or diagnostic procedure inside a subject's body. An example of such a device is an endoscope or other type of scope or insertion device, which includes a handle and an insertion portion extending distally from the handle. The insertion portion of an endoscope may be inserted into the subject's body through an opening (e.g., a natural opening or an incision) and is delivered to a site inside the body, for example, through a body lumen. The insertion portion of an endoscope or other insertion device includes a shaft that extends from outside the body to the treatment site.

During medical procedures, such as endoscopic procedures, tissue samples may be acquired from a subject's body for analysis. For example, in a biopsy procedure, tissue samples may be collected from an organ or a wall of a lumen in the body. One or more instruments may be introduced through the working channel of a scope or be fixed to the distal end of the scope to obtain the tissue samples. These tissue samples may be used to determine the presence of a pathological disorder, cancerous tissues, etc. However, biopsy procedures can be lengthy and difficult when more than one sample of tissue is necessary to make an accurate assessment, for example, when it is necessary to obtain samples of tissue from multiple locations. Furthermore, it may be cumbersome to obtain tissue samples from deep within the body at locations that are difficult to access only to remove and then reintroduce the instruments to acquire more than one sample.

### SUMMARY

This disclosure includes medical devices, systems, and related methods that may facilitate the retrieval of multiple tissue samples, for example, without the need to withdraw the device or instrument between the retrieval of each sample.

In one example, a medical device may comprise an outer tube defining a lumen and an inner tube. The inner tube may be movable within the lumen of the outer tube, and an end effector may be at a distal end of the inner tube. The end effector may retract to capture one or more tissue samples. A capsule may be positioned within a distal portion of the inner tube. The capsule may include a proximal opening and a distal opening for receiving the one or more tissue samples.

Any examples described herein may have any of these features alone or in combination. The end effector may be coupled to the distal end of the inner tube and may be configured to move between an expanded configuration and a retracted configuration for capturing and enclosing the one or more tissue samples. The capsule may further comprise a plurality of locking pins to removably couple the capsule to the inner tube. The plurality of locking pins may be positioned on opposite sides of the inner tube. The inner surface of the inner tube may include circumferential indentations to receive outer portions of the plurality of locking pins. In some aspects, the capsule may comprise an extension to removably couple the capsule to the inner tube, and a distal portion of the inner tube may include a stop surface on an inner surface of the distal portion of the inner tube. The extension may project outward from the capsule, and the extension may include a proximal end and a distal end.

The proximal end of the extension may be coupled to a portion of the capsule. The capsule may include a proximal diameter and a distal diameter, and the proximal diameter may be smaller than the distal diameter. The proximal opening of the capsule may include a filter or a sieve. The medical device may include one or more landmarks indicating an amount of a tissue sample collected within the capsule. The one or more landmarks may be radiopaque. The medical device may include a plurality of capsules, and each of the plurality of capsules may include a proximal diameter and a distal diameter. The medical device may include a proximal-most capsule and one or more distal capsules, and the proximal-most diameter of the proximal-most capsule may be smaller than the distal diameter of the proximal-most capsule. The proximal ends of the one or more distal capsules may be open allowing captured tissue samples to pass proximally.

The medical device may further comprise a handle, and the handle may include an actuator and a handle body. The handle body may be fixedly coupled to a proximal end of the outer tube. The actuator may be coupled to a proximal end of the inner tube such that movement of the actuator relative to the handle body may control movement of the inner tube relative to the outer tube. The actuator may include a suction port fluidly connected to the inner tube to apply negative pressure from the suction port through the inner tube.

In another example, a medical device may comprise an outer tube defining a lumen and an inner tube. The inner tube may be movable within the lumen of the outer tube, and an end effector may be at a distal end of the inner tube. A capsule may be positioned within a distal portion of the inner tube. The capsule may include a proximal opening and a distal opening for receiving the one or more tissue samples. The capsule may include a proximal diameter and a distal diameter, and the proximal diameter may be smaller than the distal diameter. The medical device may include a handle at a proximal end of the outer tube. The handle may include a suction port configured to apply negative pressure to introduce one or more tissue samples into the capsule. The handle may include an actuator controlling a movement of the inner tube and the end effector relative to the outer tube.

Any of the examples disclosed herein may include any of the following features in any combination. The medical device may include a plurality of capsules, and the plurality of capsules may include a proximal-most capsule and one or more distal capsules. A proximal diameter of the proximal-most capsule may be smaller than a distal diameter of the proximal-most capsule, and the proximal ends of the one or more distal capsules may be open allowing captured tissue samples to pass proximally. The plurality of capsules may further comprise a plurality of releasable connections to removably couple the plurality of capsules to the inner tube.

This disclosure also includes a method for collecting multiple tissue samples using a medical device. The method may comprise assembling a distal end of the medical device by introducing one or more capsules into a hypotube and securing the capsules; inserting the distal end of the medical device into a body lumen, organ, or cavity; positioning an end effector at a target tissue site; actuating the end effector to capture a tissue sample; applying negative pressure to move the captured tissue sample proximally into the capsule; removing the medical device from the body lumen; and extracting the one or more capsules from the hypotube by disengaging one or more releasable connections coupled to the hypotube and the one or more capsules.

According to some aspects, the one or more capsules may include a plurality of capsules. The method may further comprise repositioning the end effector to a new target site and repeating actuating the end effector and applying negative pressure to move the captured tissue sample proximally into the capsule.

### BRIEF DESCRIPTION OF THE FIGURES

The accompanying drawings, which are incorporated in and constitute a part of this specification, illustrate exemplary aspects of the disclosure and together with the description, explain the principles of the disclosure.

FIG. 1 is a side view of a medical device, according to aspects of this disclosure.

FIG. 2 illustrates a side view of the distal portion of the medical device of FIG. 1, according to aspects of this disclosure.

FIG. 3 illustrates a side view of a distal portion of another medical device, according to aspects of this disclosure.

FIGS. 4A-4C illustrate exemplary views of an assembly of a component of the distal portion of the medical device, according to aspects of this disclosure.

FIG. 5 is a block diagram depicting an exemplary method of obtaining tissue samples using a medical device, according to aspects of this disclosure.

### DETAILED DESCRIPTION

Both the foregoing general description and the following detailed description are exemplary and explanatory only and are not restrictive of the features, as claimed. As used herein, the terms “comprises,” “comprising,” “has,” “having,” “includes,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic. Additionally, terms that indicate the geometric shape of a component/surface encompass both exact and approximate shapes.

This disclosure is described with reference to exemplary medical devices, components, assemblies, and/or systems having a handle and a shaft. Although endoscopes are referenced herein, it will be appreciated that the disclosure encompasses various devices that may be inserted into a body of a subject, such as ureteroscopes, cholangioscopes, duodenoscopes, gastroscopes, endoscopic ultrasonography (“EUS”) scopes, colonoscopes, bronchoscopes, laparoscopes, arthroscopes, cystoscopes, aspiration scopes, sheaths, or catheters. Additionally, the target site may be any internal lumen, organ, cavity, or other tissue within the subject.

It should be noted that reference to any device and/or any particular procedure is provided only for convenience and is not intended to limit the disclosure. A person of ordinary skill in the art would recognize that the concepts underlying the disclosed devices and application methods may be utilized in any suitable procedure, medical or otherwise. The assemblies and systems described herein may be used in conjunction with other types of medical devices. For ease of description, portions/regions/ends of the device and/or its components are referred to as proximal and distal ends/regions. It should be noted that the term “proximal” refers to ends/regions closer to a user of the device, and the term “distal” is used herein to refer to ends/regions further away from the user. In other words, saying that a component extends “distally” indicates that a component extends in a distal direction (away from a user), and saying that a component extends “proximally” indicates that a component extends in a proximal direction (toward a user). Proximal and distal directions are labeled with arrows marked “P” and “D,” respectively, throughout various figures.

Aspects of this disclosure are now described with reference to exemplary medical devices, systems, and methods useful for collecting tissue samples, for example, biopsy samples. The medical devices herein may facilitate collection of more than one tissue sample without completely withdrawing and reintroducing the distal end of the medical device for each sample. The medical device may include an outer tube or shaft that defines a lumen, enclosing at least a portion of an inner tube or hypotube that can move within the lumen. This movement may be controlled by components on the handle of the device, allowing the user to manipulate the hypotube relative to the shaft. The handle facilitates over the movement of the hypotube, which in turn actuates an end effector to collect tissue samples. The hypotube may include a capsule or multiple capsules capable of receiving and storing tissue from a target site. In some embodiments, the hypotube may include one or more capsules. In summary, components of the hypotube and shaft may allow a user to collect multiple tissue samples, for example, cutting and storing a plurality of tissue samples within components (e.g., capsule(s)) of the medical device.

Reference will now be made in detail to examples of this disclosure described above and illustrated in the accompanying drawings. Wherever possible, the same reference numbers will be used throughout the drawings to refer to the same or like parts.

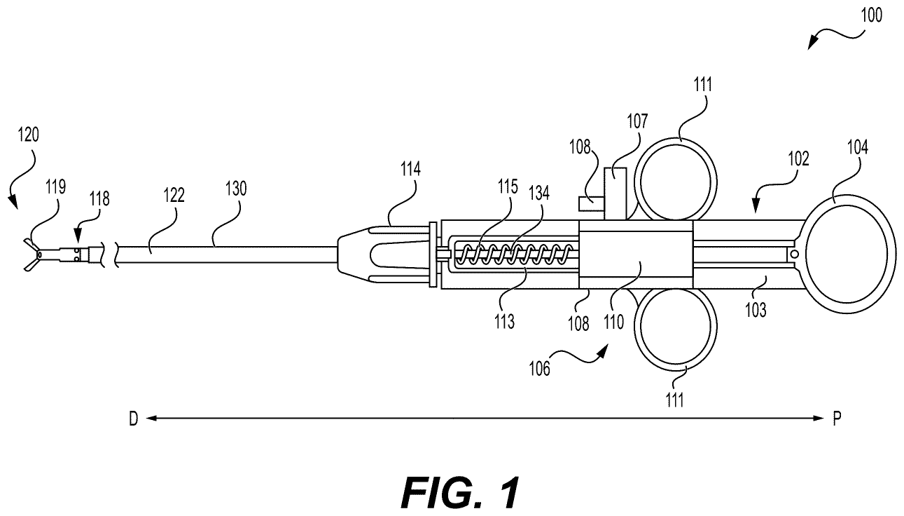

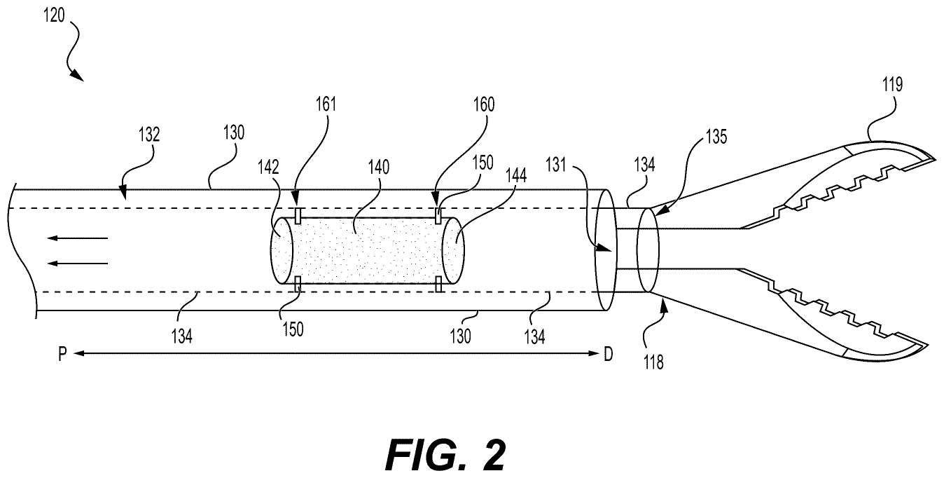

FIG. 1 illustrates a medical device 100 including a proximal end 106 and a distal end 120. Medical device 100 comprises a handle 102 and a shaft 130. In some aspects, shaft 130 includes an end effector assembly 118, for example, at distal end 120. Handle 102, positioned at proximal end 106, includes an opening or a port 107 (e.g., a biopsy port), which may be fluidly connected to a lumen 132 (FIG. 2) and/or one or more channels of shaft 130. Port 107 may also include a suction port 108, designed to connect to capital equipment, a tube, or a hose external to handle 102 that may facilitate an application of negative pressure by medical device 100. Alternatively, suction port 108 may be separate from port 107. Suction port 108 helps facilitate the application of negative pressure, or suction, by medical device 100 to capture or secure tissue samples during biopsy procedures, as will be described in further detail below. Additionally, handle 102 may include an actuator 110, which may help to actuate or otherwise control end effector assembly 118, as described in detail below. Port 107, including suction port 108, may be positioned on actuator 110 or alternatively on another portion of handle 102, for example, a handle body 103, also described in the subsequent sections.

Actuator 110 may include one or more finger slot(s) 111. For example, two finger slots 111 may extend outwardly (e.g., radially outward) from a proximal portion of actuator 110. Finger slot(s) 111 may be proximal to or otherwise adjacent to port 107. The one or more finger slot(s) 111 may be any suitable shape and size to enable a user to insert a finger through the slot 111. For example, the one or more finger slot(s) 111 may be circular (as shown), triangular, rectangular, cylindrical or any shape commonly known in the art.

Additionally, handle 102 may include handle body 103, which may be integrally formed within handle 102. Handle body 103 may include a finger ring 104 located at or near a proximal end of handle 102. Finger ring 104 may be shaped similar to or from the one or more finger slots 111 on actuator 110. Handle body 103 may include a slot, opening, or track 113, for example, extending longitudinally through a portion of handle body 103. As discussed below, a biasing element 115 (e.g., a spring or a coil) may be positioned within track 113, between the handle body 103 and the distal end of actuator 110. Alternatively, biasing element 115 may be positioned between the proximal end of actuator 110 and the handle body 103 (e.g., adjacent finger ring 104). In any of these aspects, biasing element 115 may impart a biasing force on actuator 110 toward an equilibrium position, which in turn imparts a biasing force on end effector assembly 118. For example, the biasing force on end effector assembly 118 may urge end effector assembly 118 toward the closed or retracted configuration.

Actuator 110 may be moveably coupled to handle body 103 via track 113. For example, a portion of actuator 110 may extend through (e.g., laterally) track 113, and actuator 110 may be movable along track 113. The movement of actuator 110 relative to handle body 103 may actuate or control an end effector 119, as discussed below. Actuator 110 may translate within track 113 in a first direction, towards a distal end of handle 102, and in a second direction, towards a proximal end of handle 102. The movement of actuator 110 in these directions results in the actuation or de-actuation of end effector assembly 118 (e.g., opening and closing). As discussed below, hypotube 134 may be fixedly coupled to actuator 110, such that movement of actuator 110 in a first direction or in a second direction imparts the same movement to hypotube 134.

Still referring to FIG. 1, and as noted above, medical device 100 includes shaft 130 extending from a distal end of handle 102 to distal end 120 of medical device 100. Shaft 130 may be an outer tube with a sufficient length to reach target sites within the body of a subject. Additionally, shaft 130 may be flexible enough to navigate through tortuous anatomical pathways in various body lumens. As shown in FIG. 1, handle 102 may be coupled to a proximal portion of shaft 130 via a strain relief 114. Strain relief 114 may extend from a distal end of handle 102 and overlap with a proximal portion of shaft 130, which may help to maintain the coupling of handle 102 to shaft 130. Strain relief 114 may be integrated into or connected to handle body 103 by adhesive(s), overmold, press-fit, or any other mechanisms commonly used in the art.

Hypotube 134 may be fixedly coupled to actuator 110 by various mechanisms, including adhesives, a press-fit, ultrasonic welding, and laser welding, or any other commonly known methods in the art. Hypotube 134 may extend distally through an opening in the strain relief 114 to lumen 132 of shaft 130 in such a way that both wire 116 and hypotube 134 move relative to shaft 130 within lumen 132. In other words, hypotube 134 may be translatable or slidable along lumen 132 of shaft 130. For example, distal movement of actuator 110 results in the movement of hypotube 134 in a first direction toward the distal end and in a second direction towards the proximal end. Because hypotube 134 is coupled to actuator 110, hypotube 134 (which is coupled to the end effector 119) may also move distally and proximally relative to the shaft 130. Alternatively, shaft 130 may be coupled to actuator 110, allowing shaft 130 (including end effector 119) to move distally and proximally relative to hypotube 134.

FIG. 2 depicts distal end 120 of medical device 100, including shaft 130. As discussed, shaft 130 may radially surround hypotube 134. Both hypotube 134 and shaft 130 may be composed of suitable biocompatible materials. Exemplary biocompatible materials may include, but are not limited to, Nitinol, stainless steel, and polymers, such as polyurethane, silicone, and other plastic materials. The material(s) may provide shaft 130 and/or hypotube 134 sufficient flexibility and suitable rigidity to navigate through subject anatomy. In some embodiments, hypotube 134 may be more rigid than shaft 130. For example, increased rigidity may provide additional support and stability for end effector assembly 118 during tissue collection or sampling, as described below.

Hypotube 134 may be an inner tube that is generally cylindrical, or one or more shapes that align with or partially correspond to the shape of lumen 132 of shaft 130. Hypotube 134 includes a distal opening 135 and a proximal opening (not shown) at its proximalmost end. Additionally, an end effector 119 may be coupled to or otherwise extend from distal opening 135 or a distal end of hypotube 134. Hypotube 134 may extend along the entire length of lumen 132 of shaft 130. However, the disclosure is not so limited, and the length of hypotube 134 may be adjusted based on the requirements of a medical procedure (e.g., procedures requiring more flexibility in distal end 120). For example, hypotube 134 may extend only from distal end 120 to an intermediate portion 122 (FIG. 1) of shaft 130. A longer hypotube 134 may help lumen 132 of shaft 130 maintain its shape and/or orientation as shaft 130 is guided through the body's internal passages. However, a longer hypotube 134 may make shaft 130 less flexible. In some examples where hypotube 134 does not extend an entire length of shaft 130, the proximalmost end of hypotube 134 may be coupled to one or more tubes (e.g., a tube that is more flexible than hypotube 134), and the one or more tubes may physically connect hypotube 134 to actuator 110 and/or fluidly connect hypotube 134 to suction port 108.

Still referring to FIG. 2, distal end 120 includes end effector assembly 118. End effector assembly 118 may comprise shaft 130, hypotube 134, and end effector 119. End effector 119 may be a tissue grasper, biopsy forceps, jaws, scissors, a retrieval device (such as a net or a basket), etc. End effector 119 may be fixedly coupled within the inner diameter of hypotube 134 (e.g., at distal opening 135) by adhesive, press-fit, ultrasonic welding, laser welding, or any other mechanisms commonly known in the art. In other words, end effector 119 may be fixedly coupled within the inner diameter of hypotube 134 so that end effector 119 moves simultaneously with hypotube 134 relative to shaft 130. Alternatively, end effector 119 may be fixedly coupled within the inner diameter of shaft 130 in the same manner. In this embodiment, end effector 119 may be coupled to shaft 130 so that end effector 119 moves simultaneously with shaft 130, relative to hypotube 134.

In the embodiment, end effector assembly 118 may be in a “retracted” configuration when hypotube 134 (e.g., including end effector 119) is positioned proximally to distal opening 131 of shaft 130. Biasing element 115 may exert a biasing force on actuator 110 pushing actuator 110 away (e.g., proximally) from distal end 120. The biasing force may cause hypotube 134 and consequently end effector assembly 118 to be biased towards a proximal “retracted” configuration. End effector 119 may be designed (e.g., with shape memory material) to expand when end effector 119 is not enclosed by shaft 130, and to retract when enclosed by shaft 130. As a result, end effector assembly 118 may be in an “expanded” configuration when hypotube 134 (e.g., distal opening 135 of hypotube 134) and end effector 119 are positioned distally beyond distal opening 131 of shaft 130 (FIG. 2).

In use, manipulating actuator 110 distally urges hypotube 134 in the same direction, overcoming the proximal force exerted by biasing element 115. Actuator 110 may be moved distally at least until distal opening 135 of hypotube 134 is distal to distal opening 131 of shaft 130, thereby actuating (e.g., expanding) end effector 119. Once end effector assembly 118 is in an “expanded” configuration, actuator 110 may be released allowing end effector assembly 118 to be biased proximally by biasing element 115. Alternatively, or additionally, actuator 110 may be manually moved in a proximal direction. When distal opening 135 of hypotube 134 moves proximally relative to distal opening 131 of shaft 130, end effector 119 may be urged to contract by shaft 130. Thus, actuator 110 and related mechanisms may control the transition of distal end effector assembly 118 between the expanded and retracted configurations. In addition, the user may determine the specific amount (e.g., volume) of tissue that end effector 119 may collect when actuated on a target tissue site and may manipulate actuator 110 accordingly. For example, hypotube 134 may have an inner diameter of 1.2 mm, which may affect the size of capsule 140. In other words, the inner diameter of hypotube 134 may influence the possible diameter of capsule 140. The diameter of capsule 140, in turn, affects the volume of tissue that capsule 140 may accommodate. Correspondingly, the volume of tissue that capsule 140 may accommodate affects the number of “bites” from end effector 119 that capsule 140 may receive. The number of “bites” from end effector 119 that capsule 140 may receive may also correspond to a size of end effector 119.

FIG. 2 shows distal end 120 including a capsule 140. Capsule 140 may be configured to store one or more tissue samples. Capsule 140 may be removably coupled to hypotube 134 and/or shaft 130, as will be described below. In any embodiment, capsule 140 may have a cylindrical shape and in some aspects, capsule 140 may incorporate one or more slits in an exterior, for example, a spiral cut along its length. The spiral cut may help to provide flexibility to capsule 140, enabling capsule 140 to adapt to the contours of the surrounding anatomy along with the other components of distal end 120.

Capsule 140 may include a distal opening 144 and a proximal opening 142. Distal opening 144 may be fully open, and proximal opening 142 may be partially closed. Proximal opening 142 may be partially closed via one or more mechanisms known in the art. For example, capsule 140 may include a proximal diameter and a distal diameter. The proximal diameter may be smaller than the distal diameter, such that proximal opening 142 (e.g., proximal opening 242 of capsule 240) may be smaller than distal opening 144. Alternatively, or in addition to the above, proximal opening 142 may include a filter, a sieve, mesh, lattice, or partially closed structure. In the embodiment, proximal opening 142 may be configured to help facilitate necessary negative pressure for vacuum aspiration, while preventing tissue samples from escaping, thereby maintaining sample integrity during the collection process.

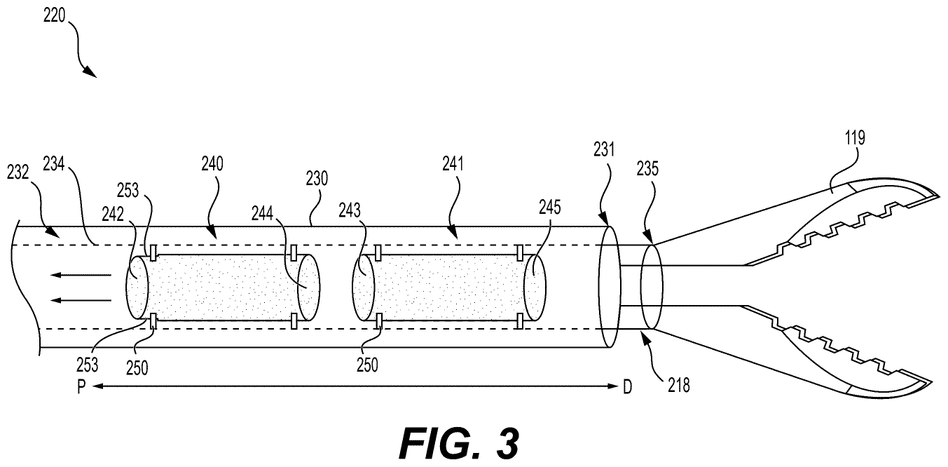

FIG. 3 shows a distal end 220, which may be similar to the previously described distal end 120, except for the differences described herein. FIG. 3 includes a shaft 230, similar to shaft 130, including a lumen 232 and a distal opening 231. Distal end 220 may also include a hypotube 234 with a distal opening 235, similar to hypotube 134. Accordingly, distal end 220 includes a distal end assembly 218, which includes an end effector 219 similar to end effector 119 in distal end assembly 218. Hypotube 234 may also be actuated proximally and distally to open and close the end effector 219. However, distal end 220 in FIG. 3 depicts additional capsules at its distal end 220, which may be adjusted based on the requirements of a medical procedure (i.e., additional capsules could be added to FIG. 2). For example, hypotube 234 includes a first capsule 240 and a second capsule 241. Capsule 140, first capsule 240, and second capsule 241, may be similar, except as described herein.

For example, in any embodiment that includes multiple capsules (e.g., FIG. 3), only the proximal-most capsule (i.e., in FIG. 3, first capsule 240) may include a proximal opening (i.e., 242) similar to proximal opening 142 (e.g., partially closed). In other words, a plurality of capsules includes a proximal outer diameter and a distal outer diameter. The proximal diameter of at least the proximal-most capsule may be smaller than distal diameter of the proximal-most capsule. As shown in FIG. 3, capsule 240 may include a tapered portion 253 such that proximal opening 242 is smaller than distal opening 244. Tapered portion 253 may help retain tissue within capsule 240. Capsules positioned distal to the proximalmost capsule may include both an open proximal opening and an open distal opening. For example, both a distal opening 245 and a proximal opening 243 of second capsule 241 may be fully open and therefore may not include a filter, a sieve, or a tapered portion 253. As shown, proximal opening 243 and distal opening 245 of second capsule 241 may be fully open and, therefore, do not include a filter, sieve, or taper. Said differently, the proximal ends of the one or more distal capsules (e.g., second capsule 241) are open to help allow captured tissue samples to pass proximally.

In other words, proximal opening 243 and distal opening 245 help facilitate vacuum aspiration proximally, and additionally, tissue sample collection to any capsule that is proximal to it, such as capsule 240. Instead, capsule 240 may include proximal opening 242 similar to proximal opening 142 (i.e., partially closed) to act as a filter, while distal opening 244 is fully open, helping to avoid interruption of the flow of negative pressure when collecting tissue samples.

Various mechanisms may be used to removably couple capsule 140 to hypotube 134, as well as capsule 240 and capsule 241 to hypotube 234. As shown in FIG. 2 and FIG. 3, capsule 140, capsule 240, and capsule 241 may be surrounded by a plurality of releasable connections, such as locking pins 150 and locking pins 250. Specifically, locking pins 150 may be removably coupled to capsule 140, while locking pins 250 may be removably coupled to capsule 240 and capsule 241. Capsule 140, capsule 240, and capsule 241 may be made of silicone, polyurethane, nitinol, or any other suitable flexible yet rigid material facilitating effective interfacing with locking pins 150 and locking pins 250.

In embodiments, capsule 140, capsule 240, and capsule 241 may include slots that interface with locking pins 150 and locking pins 250, respectively. Locking pins 150 and locking pins 250 may be similar, except some embodiments (e.g., FIG. 3) depict locking pins 250 securing additional capsules, such as capsule 240 and capsule 241. Locking pins 150 and locking pins 250 may be made of stainless steel, titanium, or nitinol. Furthermore, locking pins 150 and locking pins 250 may incorporate radiopaque materials, making them visible on various imaging modalities commonly used in the art. For example, locking pins 150 may be coated with radiopaque materials commonly used as contrasting agents, such as barium sulfate, tantalum, or platinum.

In the embodiments shown herein, each capsule (i.e., capsule 140, capsule 240, capsule 241) has four equally spaced locking pins 150 and locking pins 250, respectively. However, it will be appreciated that a greater or lesser number of locking pins 150 and locking pins 250, and unequal spacing, may be used. For example, only a single locking pin of locking pins 150 may be used. Locking pins 150 and locking pins 250 may be positioned around the inner circumference (i.e., inner surface) of hypotube 134 and hypotube 234, respectively. For example, locking pins 150 may be positioned on opposite sides of hypotube 134. The inner surface of hypotube 134 may include indentations (e.g., circumferential indentations) to receive locking pins 150. Alternatively, the indentations on the inner surface of hypotube 134 may be slots or channels, and the slots or channels may be formed via machining or another formation process.

Locking pins 150 (i.e., including locking pins 250) may be disengaged by various mechanisms. For example, a user may simply remove them after a procedure is completed. Alternatively, as discussed, hypotube 134 may be translated by a user, and as hypotube 134 moves sufficiently proximal, a threshold force may release locking pins 150 from capsule 140. Capsule 140 (i.e., including capsule 240 and capsule 241) may be aspirated out by a user at the proximal end. Additionally, or instead of the indentations, a central wire may run through locking pins 150, maintaining their engagement with capsule 140. To disengage locking pins 150, the user may actuate the wire from the proximal end, causing the locking pins 150 to retract or lose their alignment with the inner surface of hypotube 134, thereby allowing capsule 140 to be removed.

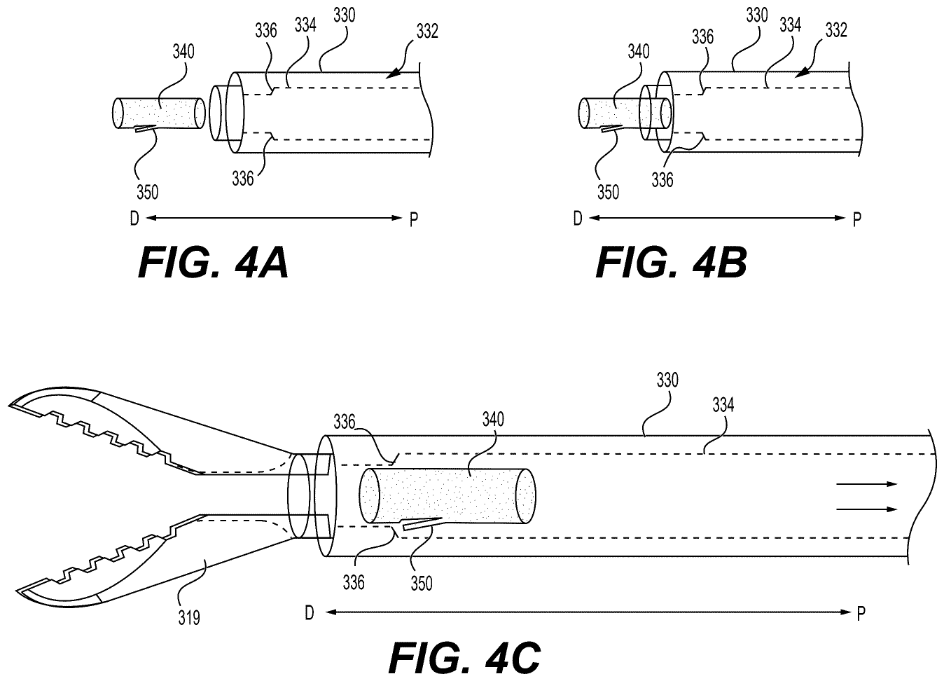

FIGS. 4A-4C depict another exemplary mechanism of removably coupling a capsule 340 to a hypotube 334. Capsule 340, shaft 330, and hypotube 334 may be similar to those described in FIG. 2 and FIG. 3, except for the releasable connection described herein. In FIG. 4A and FIG. 4B, end effector 319 is not shown coupled to hypotube 334 for merely illustrative purposes in order to depict the introduction of capsule 340 into hypotube 334. As shown in FIG. 4C, end effector 319 may be coupled to hypotube 334, similar to hypotube 134 and end effector 319.

In the embodiment depicted in FIGS. 4A-4C, capsule 340 may differ by the inclusion of an extension 350 that may serve as an alternative to locking pins 150 and locking pins 250. Extension 350 may also be used to removable couple capsule 340 to hypotube 334. Extension 350 may be integral to capsule 340 and be made of similar materials as capsule 340. Extension 350 may also be similar to locking pins 150 and locking pins 250, for example, extension 350 may also be made of plastics, silicone, flexible metals like nitinol, or any suitable material providing the necessary rigidity and flexibility for secure engagement and disengagement and include radiopaque coating. In any embodiment, extension 350 may project outward and include a proximal end that is coupled to a portion of capsule 340 and a distal end that may engage with corresponding features on hypotube 334. Extension 350 may be a flexible flap that may bend or flex under pressure, helping facilitate engagement and disengagement from a stop surface 336 of hypotube 334 as described below.

Hypotube 334 may be similar to hypotube 334 except for modifications to accommodate extension 350. For example, hypotube 334 may include a radially inward extending portion (i.e., wall) of hypotube 334 that forms a stop surface 336. Stop surface 336 may be a portion of hypotube 334 that includes a thicker wall relative to the remainder of hypotube 334. Stop surface 336 may form a smaller diameter on the distal end of hypotube 334 relative to the remainder of hypotube beyond stop surface 336. The distal end of extension 350 may engage with stop surface 336 to couple and provide a secure hold for capsule 340 to hypotube 334.

As shown in FIG. 4A, extension 350 may project in a direction opposite (e.g., distally) to the direction in which it is inserted (e.g., proximally) into hypotube 334. In the embodiment, extension 350 may bend inwardly toward capsule 340 (i.e., bending to align with a tubular shape of hypotube 334) and engage with stop surface 336 in hypotube 334 as capsule 340 is inserted. As shown in FIG. 4B, hypotube 334 begins to overlap with capsule 340 until the distal end of the extension 350 engages with a stop surface 336 on hypotube 334, as shown in FIG. 4C.

Subsequently, capsule 340 remains removably coupled within hypotube 334, similar to hypotube 134. Extension 350 may disengage from hypotube 334 in a similar manner to locking pins 150 and locking pins 250. In an embodiment, hypotube 334 may move proximally (i.e., similar to hypotube 134 and hypotube 234). The relative movement may cause extension 350 to be gradually pushed out of stop surface 336 as it is no longer aligned with the position of extension 350, allowing extension 350 to spring back to its natural position when not engaged with stop surface 336.

Referring to the embodiments depicted in FIG. 2 and FIG. 3, various aspects of this disclosure describe how capsules (e.g., capsule 140, capsule 240, capsule 241, and capsule 340) may function as landmarks to indicate the amount of tissue collected during a procedure. As discussed, the user may determine the amount of tissue collected by correlating the size of capsule 140 with the size of hypotube 134. A larger size of hypotube 134 may include a larger capsule 140, which may accommodate a greater volume of tissue sample(s) from a larger end effector 119.

In the embodiment of FIG. 2, capsule 140 may be secured by a pin segment 160 and a pin segment 161, each acting as landmarks, corresponding to a different biopsy location or a specified amount of tissue collected. For example, a user may be aware of the amount of tissue obtained by end effector 119 of distal end effector assembly 118. Each pin segment 160 and pin segment 161 may represent a predetermined amount of tissue collected, allowing a user to ascertain the amount of tissue collected at various depths. As discussed, locking pins 150 may be radiopaque helping a user monitor the position of distal end 120 and capsule 140, and the extent of tissue collection in real-time. Although capsule 140 was used as an example, first capsule 240 and second capsule 241 may also include pin segments and may be used similarly.

In the embodiment illustrated in FIG. 3, first capsule 240 and second capsule 241 may act as distinct landmarks. For example, first capsule 240 and second capsule 241 are designed to accommodate an approximate amount of tissue, which may correspond to one or more of the incremental bites taken by end effector 119 of distal end effector assembly 118. First capsule 240 may be filled by an initial depth of tissue or the known capacity for a first “bite,” and once filled (e.g., it may require multiple bites), a user may know that a specific volume of tissue has been collected from first capsule 240. Similarly, second capsule 241 may correspond to a subsequent depth of tissue.

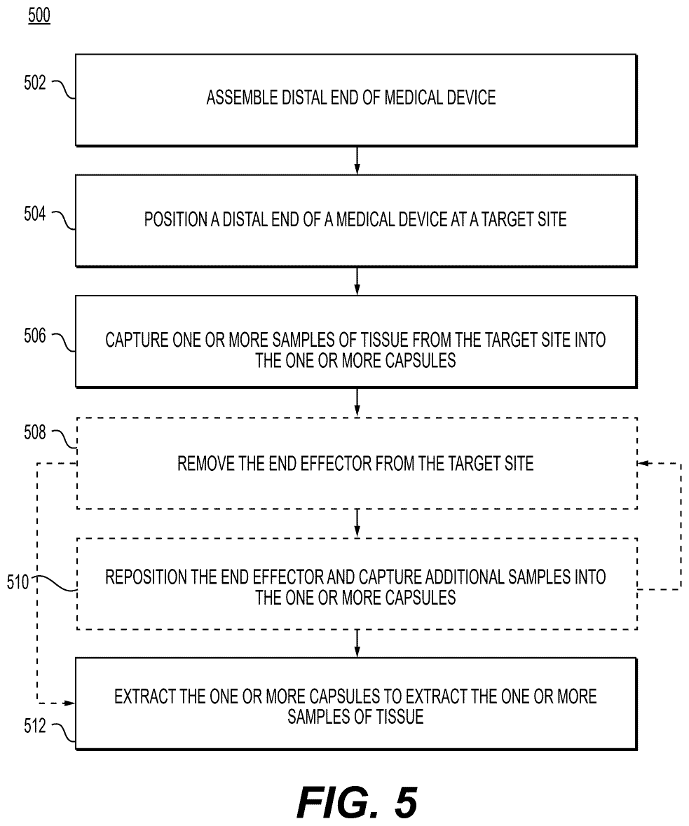

FIG. 5 illustrates a flow chart of an exemplary method 500 for a user to collect multiple tissue samples using distal end 120 of medical device 100. Method 500 may be used by a user (e.g., a medical professional) when treating a subject. For example, if distal end 120 is not assembled, a user may optionally begin with step 502 by assembling distal end 120 of medical device 100. To assemble distal end 120, a user may introduce capsule 140 and, if applicable, additional capsules (e.g., similar method is used for distal end 220 including first capsule 240 and second capsule 241). Capsule 140 may be attached using locking pins 150 (or similar releasable connection) to secure capsule 140, and if applicable, additional capsules. Furthermore, before use, the user may ensure distal end effector assembly 118 may be in a “retracted” configuration by actuating hypotube 134 (i.e., including end effector 119) to be positioned proximal to distal opening 131 of shaft 130.

Subsequently, as seen in step 504, the user may introduce assembled distal end 120 (or distal end 220 if using additional capsules) into a subject's body. This insertion may be through a lumen, organ, or other body cavity. The user then advances medical device 100 until distal end 120 is at or proximate to a target tissue site. In this example, end effector 119 is coupled to the inner circumference of hypotube 134. As will be apparent to one of ordinary skill in the art, in other examples, end effector 119 may be coupled to the inner circumference of shaft 130.

As seen in step 506, a user may capture one or more tissue samples from the target site into one or more capsules. To do so, distal end 120 of medical device 100 may be maneuvered so that a portion of tissue extends at least partially into end effector 119 of end effector assembly 118. As discussed, the combination of and connections between the aforementioned components is such that a user manipulating actuator 110 distally may translate hypotube 134 toward distal end 120, which actuates (e.g., expands) end effector 119 until end effector assembly is in an “expanded” configuration. Subsequently, distal end 120 may be maneuvered so that a portion of tissue extends at least partially into end effector 119. Then, the user may manipulate actuator 110 away from distal end 120 to de-actuate (e.g., close) end effector 119 to cut the tissue that has entered end effector 119. Afterward, actuator 110 may be further translated in a proximal direction until, for example, hypotube 134 is within shaft 130, and hypotube 134 is proximal to distal opening 131.

Still referring to step 506, handle 102 may be actuated (e.g., via a button, valve, or any suitable mechanism on actuator 110) to apply negative pressure through, for example, suction port 108. Suction port 108 may help create suction within lumen 132 of shaft 130, helping move the captured samples of tissue proximally toward an internal space of capsule 140 (or one or more capsules). Step 506 may be repeated multiple times, storing successive samples within capsule 140 until full. As discussed, the user may know the specific amount of tissue that end effector 119 can collect in a single “bite”. This known capacity allows the user to estimate the total volume of tissue that can be sequentially stored within capsule 140. A user may reapply negative pressure to move each new tissue sample into capsule 140. Optionally, the user may use an imaging mechanism to evaluate the volume and depth of tissue collected by identifying pin segment 160 and pin segment 161 during a procedure.

The user may then proceed to step 508 and remove end effector 119 from the target site and choose to proceed to step 510 or optionally proceed directly to step 512. In step 512, the user may remove distal end 120 of medical device 100 from the body lumen, and capsule 140 may be extracted from distal end 120 in one or more ways described above regarding the disengagement of locking pins 150 or locking pins 200, or in the manner described for capsule 340.

Alternatively, the user may proceed to step 510 (similar to step 506) if additional tissue samples are needed. In step 510, without removing distal end 120 from the body lumen, the user may reposition end effector 119 to a new target site (or a different portion of the original target site) and actuate end effector 119 to obtain additional tissue samples. Subsequently, the user may proceed to step 512 to remove the device and extract capsule 140, or alternatively, repeat step 508 until the user has collected the desired amount of tissue samples. As mentioned, a similar method may be employed for distal end 220 including first capsule 240 and second capsule 241.

The medical devices herein may facilitate the retrieval of multiple tissue samples without the need to withdraw the medical device between the retrieval of each sample. Further, the medical devices herein may help to reduce the duration of procedures to collect multiple tissue samples and/or may reduce the number of times a user may have to insert a medical instrument and find a difficult-to-access tissue site.

We claim:

1. A medical device, comprising:
an outer tube defining a lumen;
an inner tube movable within the lumen of the outer tube;
an end effector at a distal end of the inner tube, wherein the end effector retracts to capture one or more tissue samples; and
a capsule positioned within a distal portion of the inner tube, wherein the capsule includes a proximal opening and a distal opening for receiving the one or more tissue samples.

2. The medical device of claim 1, wherein the end effector is coupled to the distal end of the inner tube and is configured to move between an expanded configuration and a retracted configuration for capturing and enclosing the one or more tissue samples.

3. The medical device of claim 1, wherein the capsule further comprises a plurality of locking pins to removably couple the capsule to the inner tube.

4. The medical device of claim 3, wherein the plurality of locking pins is positioned on opposite sides of the inner tube.

5. The medical device of claim 3, wherein an inner surface of the inner tube includes circumferential indentations to receive outer portions of the plurality of locking pins.

6. The medical device of claim 1, wherein the capsule further comprises an extension to removably couple the capsule to the inner tube, and a distal portion of the inner tube includes a stop surface on an inner surface of the distal portion of the inner tube.

7. The medical device of claim 6, wherein the extension projects outward from the capsule, and wherein the extension includes a proximal end and a distal end, wherein the proximal end of the extension is coupled to a portion of the capsule.

8. The medical device of claim 1, wherein the capsule includes a proximal diameter and a distal diameter, wherein the proximal diameter is smaller than the distal diameter.

9. The medical device of claim 1, wherein the proximal opening of the capsule includes a filter or a sieve.

10. The medical device of claim 1, wherein the medical device includes one or more landmarks indicating an amount of a tissue sample collected within the capsule.

11. The medical device of claim 10, wherein the one or more landmarks are radiopaque.

12. The medical device of claim 1, wherein the medical device includes a plurality of capsules, wherein each of the plurality of capsules include a proximal diameter and a distal diameter.

13. The medical device of claim 12, wherein the medical device includes a proximal-most capsule and one or more distal capsules, wherein the proximal diameter of the proximal-most capsule is smaller than the distal diameter of the proximal-most capsule.

14. The medical device of claim 13, wherein the proximal ends of the one or more distal capsules are open allowing captured tissue samples to pass proximally.

15. The medical device of claim 1, further comprising a handle, wherein the handle includes an actuator and a handle body, wherein the handle body is fixedly coupled to a proximal end of the outer tube, wherein the actuator is coupled to a proximal end of the inner tube such that movement of the actuator relative to the handle body controls movement of the inner tube relative to the outer tube, and wherein the actuator includes a suction port fluidly connected to the inner tube to apply negative pressure from the suction port through the inner tube.

16. A medical device, comprising:
an outer tube defining a lumen;
an inner tube movable within the lumen of the outer tube;
an end effector at a distal end of the inner tube;
a capsule positioned within a distal portion of the inner tube, wherein the capsule includes a proximal opening and a distal opening for receiving tissue samples, wherein the capsule includes a proximal diameter and a distal diameter, wherein the proximal diameter is smaller than the distal diameter; and
a handle at a proximal end of the outer tube, the handle including:
a suction port configured to apply negative pressure to introduce one or more tissue samples into the capsule; and
an actuator controlling a movement of the inner tube and the end effector relative to the outer tube.

17. The medical device of claim 16, wherein the medical device includes a plurality of capsules, wherein the plurality of capsules includes a proximal-most capsule and one or more distal capsules, wherein a proximal diameter of the proximal-most capsule is smaller than a distal diameter of the proximal-most capsule, and wherein the proximal ends of the one or more distal capsules are open allowing captured tissue samples to pass proximally.

18. The medical device of claim 17, wherein the plurality of capsules further comprises a plurality of releasable connections to removably couple the plurality of capsules to the inner tube.

19. A method for collecting multiple tissue samples using a medical device, comprising:
assembling a distal end of the medical device by introducing one or more capsules into a hypotube and securing the capsules;
inserting the distal end of the medical device into a body lumen, organ, or cavity;
positioning an end effector at a target tissue site;
actuating the end effector to capture a tissue sample;
applying negative pressure to move the captured tissue sample proximally into the capsule;
removing the medical device from the body lumen; and
extracting the one or more capsules from the hypotube by disengaging one or more releasable connections coupled to the hypotube and the one or more capsules.

20. The method of claim 19, wherein the one or more capsules includes a plurality of capsules and wherein the method further comprises repositioning the end effector to a new target site and repeating actuating the end effector and applying negative pressure to move the captured tissue sample proximally into the capsule.
